(19) United States
(12) Patent Application Publication
(10) Pub. No.: US 2025/0281725 A1
(43) Pub. Date: Sep. 11, 2025

(54) GUIDE WIRE

(71) Applicant: ABIOMED Europe GmbH, Aachen (DE)

(72) Inventors: Katrin Lunze, Aachen (DE); Christoph Nix, Aachen (DE); Thorsten Siess, Aachen (DE)

(21) Appl. No.: 18/862,027

(22) PCT Filed: May 2, 2023
(86) PCT No.: PCT/EP2023/061462

§ 371 Date: Oct. 31, 2024

(30) Foreign Application Priority Data
May 4, 2022 (DE) 10 2022 204 399.5

(51) Int. Cl. A61M 25/09

(52) U.S. Cl. CPC A61M 25/09; A61M 2205/3317

(57) ABSTRACT

The present invention relates to a guidewire (10) for placing a medical device into a patient's body, the guidewire (10) comprising: an elongated main body (12) having a distal axial end (14) and a proximal axial end (16). The elongated main body (12) comprises a first portion (18) having a first stiffness and a second portion (20) having a second stiffness. The first stiffness of the first portion (18) is different from the second stiffness of the second portion (20).

The present invention relates to a guidewire for placing a medical device into a patient's body.

### BACKGROUND OF THE INVENTION

A medical device, such as an intravascular blood pump, an intracardiac blood pump or a catheter, can be introduced into a patient in various ways. Such medical devices are generally introduced surgically or percutaneously during a cardiac procedure through the vascular system. In one common approach, the medical devices are inserted through the femoral artery or axillary artery using a guidewire. The guidewire can be inserted into the respective artery through an arteriotomy to create an insertion path for the medical device. As such, the guidewire is advanced through the respective artery until the distal axial end of the guidewire is deployed within the left ventricle or the right ventricle respectively. A portion of the medical device is then advanced along the guidewire until it is correctly placed in the respective ventricle. Once the medical device has been inserted, the guidewire is excerpted from the patient's body.

Further, in case the placed medical devices need to be replaced, a guidewire is again advanced along the medical device to be replaced until its distal axial end is correctly positioned within the patient's heart. Thereafter the medical device is excerpted from the patient's body, wherein the guidewire remains stationary. A new medical device is then advanced along the guidewire as described above.

In case the medical device is a blood pump, different types are known from the prior art and are intended to support the function of a patient's heart, either in a short-term application, in which the blood pump is placed in the patient for a couple of days or weeks, or in a long-term application, in which the blood pump is placed in the patient's heart for a couple of weeks or months.

In general, a blood pump is intended to unload blood from the patient's heart into a vessel to provide hemodynamic support. When placed in the left ventricle of the patient's heart, the blood pump pumps blood, for example from the left ventricle of the heart and delivers blood, for example into the aorta. When placed in the right ventricle of the patient's heart, the blood pump pumps blood from the inferior vena cava, bypasses the right atrium and right ventricle, and delivers blood into the pulmonary artery.

During planned surgery, the guidewire may be introduced into the patient's body using fluoroscopy to ensure a correct positioning of guidewire and hence, of the medical device in the patient's heart. Commonly, X-ray is used for fluoroscopy.

However, there are certain limitations and side effects associated with fluoroscopy and, in particular, with X-ray. During planned surgery for placing the medical device or the guidewire respectively X-ray is not available without restrictions. In addition, the patient and the physician are exposed to a high amount of radiation when conducting X-ray. When it comes to an emergency condition making it necessary to immediately introduce a medical device into the patient's body, X-ray is often not available, e.g. at an Intensive Care Unit or in an ambulance car.

Furthermore, correctly placing the distal axial end of the guidewire in the patient's heart requires some practice and interventional skills by the physician. In particular, it needs to be ensured that the guidewire neither perforates a blood vessel during placement, nor enters a non-intended blood vessel, such as the coronary arteries.

Therefore, the need exists to provide an ameliorated guidewire for facilitating the placement of a medical device into a patient's body.

### SUMMARY OF THE INVENTION

According to a first aspect, a guidewire for placing a medical device into a patient's body comprises an elongated main body having a distal axial end and a proximal axial end. The elongated main body comprises a first portion having a first stiffness and a second portion having a second stiffness. The first stiffness of the first portion is different from the second stiffness of the second portion.

Accordingly, the first stiffness of the first portion can be chosen to be flexible enough to ensure that a perforation of the blood vessel is inhibited. Further, the first stiffness of the first portion can be chosen to be suitable for passing e.g., the aortic valve when introducing the guidewire into the patient's body. The second stiffness of the second portion may be chosen to be stiff enough for delivering the medical device and for advancing the guidewire within the patient's body.

Preferably, the first portion extends from the distal axial end in a direction of the proximal axial end. The second portion may be adjacent to the first portion and may extend in a direction of the proximal axial end. The second portion may be directly adjacent to the first portion or may be in close vicinity. Hence, the term “adjacent” does not necessarily mean that there is direct contact between the first portion and the second portion, but an intermediate portion may be in between the first portion and the second portion. Thus, the first portion may be the tip portion of the guidewire and the second portion may compose the remainders of the guidewire. Of course, a third portion having a different stiffness compared to the first portion and/or the second portion may be provided adjacent the second portion extending in the direction of the proximal axial end.

Preferably, the first stiffness comprises a first axial stiffness and the second stiffness comprises a second axial stiffness, wherein the first axial stiffness is different from the second axial stiffness. This ensures that a suitable stiffness can be chosen for the first portion preventing a perforation of a blood vessel. On the other hand, a sufficient stiffness can be chosen for the second portion allowing for force transfer to push the guidewire forward.

Preferably, the first axial stiffness is smaller than the second axial stiffness. Hence, the first axial stiffness may be in a range of 1.4 mN/mm to 30 mN/mm. The second axial stiffness may be in a range of 1.4 mN/mm to 30 mN/mm. The second axial stiffness is sufficiently high to allow for pushing the guidewire forward without the risk of wave formation. The first axial stiffness is sufficiently low to prevent perforation of a blood vessel while pushing the guidewire forward. Alternatively, the first axial stiffness may be substantially identical to the second axial stiffness.

Preferably, the first stiffness comprises a first lateral stiffness and the second stiffness comprises a second lateral stiffness, wherein a range of the first lateral stiffness is different from a range of the second lateral stiffness. Preferably, the range of the first lateral stiffness is 20 mN/mm to 3.00 N/mm, preferably 30 mN/mm to 2.30 N/mm and highly preferably 39 mN/mm to 2.28 N/mm. Preferably, the range of the second lateral stiffness is 1.00 N/mm to 3.50 N/mm, preferably 1.50 N/mm to 3.00 N/mm and highly preferably 1.75 N/mm to 2.28 N/mm. This ensures a sufficiently high degree of bending capability but further reduces the risk of perforation of a blood vessel when pushing the guidewire forward.

The first portion preferably has an axial extension of up to 15 cm. The total axial extension between the distal axial end and the proximal axial end is preferably in a range of 120 cm to 260 cm.

Preferably, the first portion comprises an atraumatic tip extending from the distal axial end. The atraumatic tip is preferably a J-tip. The radius of the J-tip is preferably chosen to be large enough to not enter non-intended blood vessels when pushing the guidewire forward e.g., the coronary arteries. Therefore, the atraumatic tip preferably has a axial stiffness in the range of 1.4 mN/mm to 30 mN/mm and preferably a lateral stiffness of 39 mN/mm to 353 mN/mm.

In addition, when setting the first axial stiffness and/or the first lateral stiffness in the ranges according to the disclosure and/or providing an atraumatic tip, there is no need for a pigtail at the distal axial end of the guidewire for crossing the aortic valve.

The guidewire may comprise a sensor, wherein the sensor may be disposed in or next to the first portion, wherein the sensor may preferably be disposed in a region between the atraumatic tip and the second portion. Alternatively, the sensor may be integrated in the atraumatic tip. The sensor is preferably a tracking sensor. Preferably, the sensor is an electromagnetic tracking sensor. Thus, the position of the sensor and accordingly also of the first portion of the guidewire can be tracked within the patient's body by using a suitable tracking device e.g., an electromagnetic field generator. This gives a further aid to the physician when placing the guidewire in the patient's body. Preferably, the guidewire further comprises a lead arrangement. The lead arrangement may be attached to the sensor and the lead arrangement may extend in axial direction to the proximal axial end of the main body. The signals of the sensor can be communicated or transferred respectively with the lead arrangement.

Preferably, the main body comprises a hollow portion and the lead arrangement may be disposed within the hollow portion. The lead arrangement is secured within the hollow portion of the main body and may not tear due to bending or catching.

Preferably, the main body may comprise a stabilizing coil, wherein the lead arrangement is wound in parallel to the stabilizing coil. This also inhibits tearing of the lead arrangement.

Preferably, the sensor is disposed in a magnetically permeable housing, the housing preferably comprises a plastic material. The housing protects the sensor from damage and further allows for signal continuity.

Alternatively, the sensor may be disposed within a protection layer, preferably an extruded protection layer or coating. The protection layer is preferably magnetically permeable. The protection layer protects the sensor from damage and further allows for continuity. Further, the entire main body of the guidewire may be coated with the protection layer.

The guidewire may comprise at least one further sensor. The at least one further sensor may be disposed at a certain distance from the distal end to the proximal end or a certain distance from the sensor disposed in the region between the atraumatic tip and the second portion or in the atraumatic tip. Preferably, the certain distance may be 10 cm. The guidewire may comprises a plurality of sensors distributed along the guidewire. The sensors may be equally spaced from each other in the axial direction, e.g. by the certain distance. The sensors may also be non-equally spaced from each other in the axial direction.

Preferably, the sensor comprises a measurement coil, wherein the measurement coil is directly wound onto a portion of the guidewire, preferably onto a portion of the main body, preferably onto the hollow portion of the main body. Preferably, the at least one further sensor comprises a measurement coil, wherein the measurement coil is directly wound onto a portion of the guidewire, preferably onto a portion of the main body, preferably onto the hollow portion of the main body. This could also be done at several locations of the guidewire. Further, the whole portion is then covered to prevent rupture of the lead arrangement. Preferably, the main body comprises at least one cardiac echo marker. This allows to track the position of the guidewire by medical ultrasound or sonography.

The medical device according to the present disclosure may correspond to the aforementioned intravascular blood pump, the intracardiac blood pump or the catheter.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the drawings:

FIG. 1 shows a schematic side view of a guidewire according to a first embodiment;

FIG. 2 shows a schematic side view of a guidewire according to a second embodiment;

FIG. 3 shows a schematic side view of a guidewire according to third embodiment; and

FIG. 4 shows a schematic side view of a guidewire according to fourth embodiment.

### DETAILED DESCRIPTION

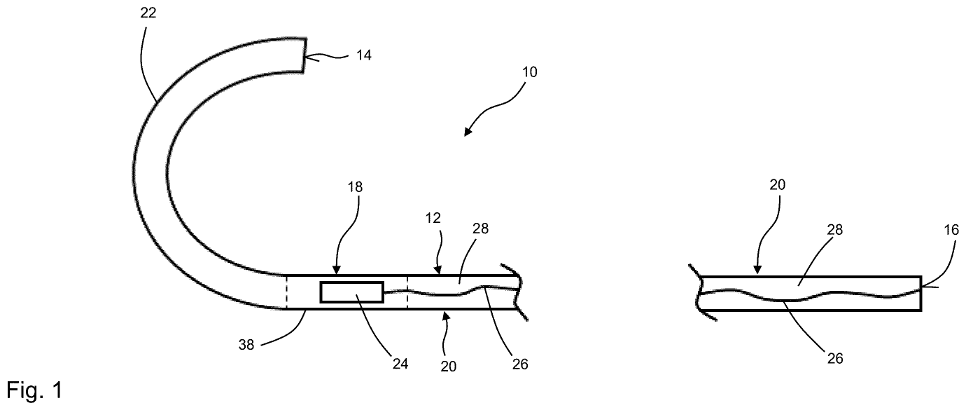

A schematic side cross sectional view of a guidewire 10 for placing a medical device in a patient's body according to a first embodiment is shown in FIG. 1. The guidewire 10 comprises an elongated main body 12 having a distal axial end 14 and a proximal axial end 16. The main body 12 has a virtually cylindrical shape and an overall length of 120 cm to 260 cm, i.e. the total axial extension between the distal axial end 14 and the proximal axial end 16 is 120 cm to 260 cm.

The main body 12 has a first portion 18 extending from the distal axial end 14 towards the proximal axial end 16 and a second portion 20 extending from the end of the first portion 18 to the proximal axial end 16. The first portion 18 has an overall length of 15 cm and comprises an atraumatic tip 22 in form of a J-tip. The J-tip 22 has a length of about 8 cm and ends at the dashed line on the left shown in FIG. 1. The J-tip 22 could be made out of a plastic material or out of a metal coil. The border between the first portion 18 and the second portion 20 is denoted by the right dashed line in FIG. 1. The J-tip 22 ensures that the guidewire 10 does not enter small vasculature such as the coronary arteries when pushed forward into the patient's body or heart respectively. Thus, the diameter of the J-tip 22 has to be sufficiently large to not enter the coronary arteries.

The guidewire 10 may have an outer diameter of 0.025″ (approximately 0.0635 cm) or smaller and has an outer sheath 38 made of a highly biocompatible plastic material such as PTFE or any other hydrophilic material. The outer sheath 38 defines a hollow portion 28 within the main body 12 of the guidewire 10.

A sensor 24 is disposed within the hollow portion 28 of the main body 12. In particular, the sensor 24 is disposed in the first portion 18 in a region between the end of the J-tip 22 and the end of the first portion 18, i.e. in a region of 8 cm to 15 cm from the distal axial end 14.

A lead arrangement 26 is attached to the sensor 24 and extends within the hollow portion 28 to the proximal axial end 16. For simplicity reasons, the lead arrangement 26 is shown as a line, but it may comprise of two or more twisted or untwisted leads. In addition the lead arrangement 26 may be covered or coated e.g., with a PTFE coating. The sensor 24 may be an electromagnetic tracking sensor and the position of the sensor 24 may be tracked by a suitable device e.g., an electromagnetic field generator. The sensor 24 may be a sensor supporting position measurements with six degrees of freedom or with five degrees of freedom. The signals of the sensor 24 are transmitted via the lead arrangement 26 to a suitable device in a known manner.

The first portion 18 has a first stiffness comprising of a first lateral stiffness and first axial stiffness. Here, lateral stiffness denotes the stiffness in the radial direction of the main body 12 and axial stiffness denotes the stiffness in the axial direction of the main body 12. The second portion 20 has a second stiffness comprising of a second lateral stiffness and a second axial stiffness.

The first axial stiffness of the first portion 18 is smaller than the first axial stiffness of the second portion 20. In particular, the first axial stiffness of the first portion 18 is in a range of 1.4 mN/mm to 30 mN/mm. In particular, the J-tip 22 of the first portion 18 has a axial stiffness in the range of 1.4 mN/mm to 30 mN/mm and the remainders of the first portion 18 has first axial stiffness of in the range of 1.4 mN/mm to 30 mN/mm. The second axial stiffness of the second portion is in a range of 1.4 mN/mm to 30 mN/mm.

The range of the first lateral stiffness of the first portion 18 is also different from the range of the second lateral stiffness of the second portion 20. The range of the first lateral stiffness of the first portion 18 is 39 mN/mm to 2.28 N/mm and the range of the second lateral stiffness of the second portion 20 is 1.75 N/mm 2.28 N/mm.

Further, the first lateral stiffness of the first portion 18 is not homogenous along the axial extension of the first portion 18. The J-tip 22 has a lateral stiffness of 39 mN/mm to 353 mN/mm. The remaining part of the first portion 18 has a first lateral stiffness of 255 mN/mm to 2.28 N/mm, whereas a gradient may be formed from the end of the J-tip 22 to the end of the first portion 18 with a gradually increasing first lateral stiffness.

This ensures that the guidewire 10 can be advanced through the blood vessel without forming waves and without the danger of perforating the blood vessel. Further, the reduces stiffness of the first portion 18 compared to the stiffness of the second portion 20 ensures that the guidewire can cross the aortic valve without perforation

In this embodiment, the varying stiffness can be achieved by varying the material composition of the outer sheath 38 in the axial direction between the distal axial end 14 and the proximal axial end 16. Further, a support structure may be provided to vary the stiffness between the first portion and the second portion. The support structure may be embedded in the outer sheath 38 or may be positioned radially inwardly of the outer sheath 38.

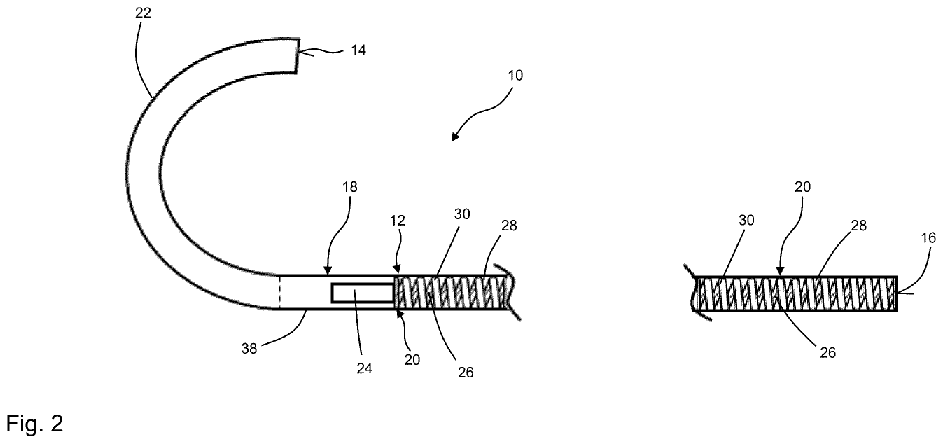

Further, a varying stiffness can also be achieved by using a stabilizing coil 30 which is disposed in the hollow portion 28 of the main body 12 of the guidewire 10, as shown in the second embodiment of the present disclosure as depicted as a schematic side cross sectional view in FIG. 2. Of course, the stabilizing coil 30 could also be placed outside of the hollow portion 28.

In the second embodiment, the stabilizing coil 30 is disposed in the second portion 20 and extends from the end of the first portion 18 to the proximal axial end 16. The stabilizing coil 30 increases the stiffness of the second portion 20, i.e. the second axial stiffness and the second lateral stiffness. As such, the material composition of the outer sheath 38 can be chosen to be homogenous, which thus results in different first lateral stiffness and first axial stiffness compared to the second lateral stiffness and the second axial stiffness due to the stabilizing coil 30. The first lateral stiffness and the first axial stiffness of the first portion 18 can be in the ranges as denoted above in terms of the first embodiment. Accordingly, the second lateral stiffness and the second axial stiffness can also be in the ranges as denoted above in terms of the first embodiment.

The second embodiment of the guidewire shown in FIG. 2 also differs from the guidewire according to the first embodiment shown in FIG. 1 in that the lead arrangement 26 is wound in parallel to the coils of the stabilizing coil 30. As shown in FIG. 2, the lead arrangement 26 may be guided to be radially inwardly of the coils of the coil arrangement 26. Of course, it is also possible to have the lead arrangement 26 guided between the coils of the stabilizing coil 30. Albeit, the lead arrangement 26 is coated with a protective coating to prevent tearing of the lead arrangement caused by e.g., bending of the guidewire 10 during placement of the same.

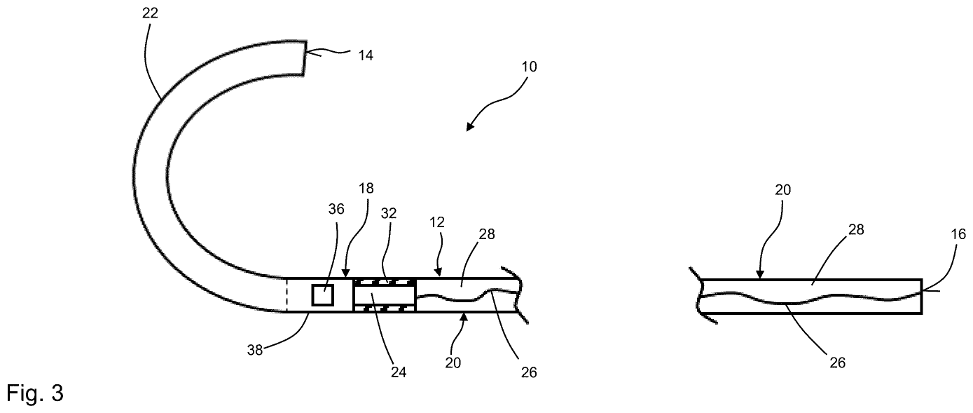

A schematic side cross sectional view of a third embodiment of a guidewire 10 is shown in FIG. 3. The guidewire 10 according to the third embodiment differs from the guidewire according to the first embodiment in that the sensor 24 is disposed within a housing 32. The housing 32 is composed of a magnetically permeable material, e.g., a plastic material like PTFE or silicone. This ensures that electromagnetic tracking of the sensor 24 is possible.

The housing 32 may either be disposed within the hollow portion 28 and hence, radially inwardly of the outer sheath 38 in the first portion 18. Alternatively, the housing 32 may be configured as an intermediate portion between the first portion 18 and the second portion 20 in the axial direction. The first portion 18 and the second portion 20 may be molded to the housing 32. Further, the housing 32 may be provided as an extruded protection layer.

In addition, the first portion 18 comprises a cardiac echo marker 36. The cardiac echo marker 36 is composed of a material not permeable to ultrasonic waves e.g., a ceramic material. In addition, the cardiac echo marker 36 may have a specific shape which allows the physician to identify the cardiac echo marker 36 when using medical ultrasound or sonography. Accordingly, the echo marker 36 delivers further information regarding the position of the first portion 18 within the patient's body. Of course, the guidewire 10 may comprise more than one echo marker 36 distributed along the axial extension of the main body 12 of the guide wire 10.

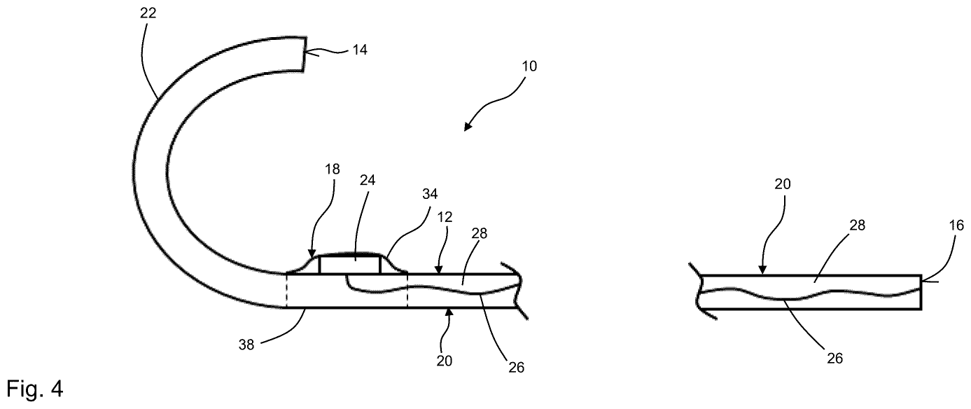

A schematic side cross sectional view of a guidewire 10 for placing a medical device in a patient's body according to a fourth embodiment is shown in FIG. 4. The guidewire 10 according to the fourth embodiment differs from the guidewire according to the first embodiment in that the sensor 24 is not disposed within the hollow portion 28. Rather, the sensor 24 is positioned radially outwardly of the outer sheath 38 on the outer peripheral surface of the first portion 18. The sensor 24 is protected by a housing 34 in form of a protective layer or coating.

Of course, the individual features of the embodiments as described above can be combined. For instance, the echo marker 36 may also be provided in a guidewire 10 according to the first embodiment, the second embodiment or the fourth embodiment. As such, the stabilizing coil 30 can also be used with a guidewire according to e.g., the third embodiment or the fourth embodiment.

### Exemplary Implementations

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:
A1 Guidewire for placing a medical device into a patient's body, the guidewire comprising: an elongated main body having a distal axial end and a proximal axial end; wherein the elongated main body comprises a first portion having a first stiffness and a second portion having a second stiffness, wherein the first stiffness of the first portion is different from the second stiffness of the second portion.
A2 Guidewire according to paragraph A1, wherein the first portion extends from the distal axial end in a direction of the proximal axial end
A3 Guidewire according to paragraph A1 or A2, wherein the second portion is adjacent to the first portion and extends in a direction of the proximal axial end.
A4 Guidewire according to any one of the preceding paragraphs A1 to A3, wherein the first stiffness comprises a first axial stiffness and the second stiffness comprises a second axial stiffness, wherein the first axial stiffness is different from the second axial stiffness.
A5 Guidewire according to paragraph A4, wherein the first axial stiffness is smaller than the second axial stiffness.

A6 Guidewire according to paragraph A4 or A5, wherein the first axial stiffness is in a range of 1.4 mN/mm to 30 mN/mm and/or wherein the second axial stiffness is in a range of 1.4 mN/mm to 30 mN/mm.
A7 Guidewire according to any one of the preceding paragraphs A1 to A6, wherein the first stiffness comprises a first lateral stiffness and the second stiffness comprises a second lateral stiffness, wherein a range of the first lateral stiffness is different from a range of the second lateral stiffness.
A8 Guidewire according to paragraph A7, wherein the range of the first lateral stiffness is 20 mN/mm to 3.00 N/mm, preferably 30 mN/mm to 2.30 N/mm and highly preferably 39 mN/mm to 2.28 N/mm.
A9 Guidewire according to paragraph A7 or A8, wherein the range of the second lateral stiffness is 1.00 N/mm to 3.50 N/mm, preferably 1.50 N/mm to 3.00 N/mm and highly preferably 1.75 N/mm to 2.28 N/mm.
A10 Guidewire according to any one of the preceding paragraphs A1 to A9, wherein the first portion has an axial extension of up to 15 cm.
A11 Guidewire according to any one of the preceding paragraphs A1 to A10, wherein the elongated main body had an axial extension of 120 cm to 260 cm.
A12 Guidewire according to any one of the preceding paragraphs A1 to A11, wherein the first portion comprises an atraumatic tip extending from the distal axial end.
A13 Guidewire according to paragraph A12, wherein the atraumatic tip is a J-tip.
A14 Guidewire according to paragraph A12 or A13, wherein the atraumatic tip has a axial stiffness in the range of 1.4 mN/mm to 30 mN/mm
A15 Guidewire according to paragraph A13 or A14, wherein the atraumatic tip has a lateral stiffness of 39 mN/mm to 353 mN/mm.
A16 Guidewire according to any one of the preceding paragraphs A1 to A15, wherein the guidewire further comprises a sensor.
A17 Guidewire according to paragraph A16, wherein the sensor is disposed in or next to the first portion.
A18 Guidewire according to paragraph A16 or A17, wherein the sensor is disposed in a region between the atraumatic tip and the second portion.
A19 Guidewire according to any one of the preceding paragraphs A16 to A18, wherein the sensor is disposed in a magnetically permeable housing
A20 Guidewire according to paragraph A19, wherein the housing comprises a plastic material.
A21 Guidewire according to any one of the preceding paragraphs A16 to A20, wherein the sensor is disposed within an extruded protection layer.
A22 Guidewire according to any one of the preceding paragraphs A16 to A21, wherein the guidewire further comprises a lead arrangement, wherein the lead arrangement (26) is attached to the sensor
A23 Guidewire according to paragraph A22, wherein the lead arrangement extends in axial direction to the proximal axial end of the main body
A24 Guidewire according to paragraph A22 or A23, wherein the lead arrangement is preferably coated or covered.
A25 Guidewire according to any one of the preceding paragraphs A1 to A24, wherein the main body comprises a hollow portion.
A26 Guidewire according to paragraph A25, wherein the lead arrangement is disposed within the hollow portion.
A27 Guidewire according to any one of the preceding paragraphs A1 to A26, wherein the main body comprises a stabilizing coil.
A28 Guidewire according to paragraph A27, wherein the lead arrangement is wound in parallel to the stabilizing coil.
A29 Guidewire according to any one of the preceding paragraphs A16 to A28, wherein the guidewire comprises a at least one further sensor,
A30 Guidewire according to paragraph A29, wherein the guidewire comprises a plurality of further sensors.
A31 Guidewire according to paragraph A29 or A30, wherein the at least one further sensor is disposed at a certain distance from the distal end to the proximal end.
A32 Guidewire according to paragraph A29 or A30, wherein the at least one further sensor is disposed at a certain distance from the sensor disposed in the region between the atraumatic tip and the second portion or in the atraumatic tip.
A33 Guidewire according to paragraph A31 or A32, wherein the certain distance is 10 cm.
A34 Guidewire according to any one of the preceding paragraphs A29 to A33, wherein the lead arrangement (26) is attached to the at least one further sensor.
A35 Guidewire according to any one of the preceding paragraphs A29 to A34, wherein the at least one further sensor is disposed in a magnetically permeable housing, the housing preferably comprises a plastic material.
A36 Guidewire according to any one of the preceding paragraphs A29 to A35, wherein the at least one further sensor is disposed within an extruded protection layer. A37 Guidewire according to any one of the preceding paragraphs A16 to A36, wherein the sensor comprises a measurement coil, wherein the measurement coil is directly wound onto a portion of the guidewire, preferably onto a portion of the main body, preferably onto the hollow portion of the main body.
A38 Guidewire according to paragraph A37, wherein the at least one further sensor comprises a measurement coil, wherein the measurement coil is directly wound onto a portion of the guidewire, preferably onto a portion of the main body, preferably onto the hollow portion of the main body
A39 Guidewire according to any one of the preceding paragraphs A1 to A28, wherein the main body comprises at least one cardiac echo marker.

As utilized herein, the terms “approximately”, “about”, “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure. The terms “at least partially” or “partially” as used herein mean both partial and entirely or complete respectively.

Herein, “proximal” and “distal” are seen relative to the medical staff or physician. Thus, proximal designates something which is relatively close to the physician whereas distal designates something which is relatively far away from the physician when the guidewire is introduced into the patient's body.

### LIST OF REFERENCE SIGNS

10 guidewire
12 main body
14 distal axial end
16 proximal axial end
18 first portion
20 second portion
22 atraumatic tip/J-tip
24 sensor
26 lead arrangement
28 hollow portion
30 stabilizing coil
32 housing
34 housing/coating
36 cardiac echo marker
38 outer sheath

1. A guidewire for placing a medical device into a patient's body, the guidewire comprising:
an elongated main body having a distal axial end and a proximal axial end,
wherein the elongated main body comprises a first portion having a first stiffness and a second portion having a second stiffness, and
wherein the first stiffness of the first portion is different from the second stiffness of the second portion.

2. The guidewire according to claim 1, wherein
the first portion extends from the distal axial end in a direction of the proximal axial end, and
wherein the second portion is adjacent to the first portion and extends in a direction of the proximal axial end.

3. The guidewire according to claim 1, wherein
the first stiffness comprises a first axial stiffness and the second stiffness comprises a second axial stiffness, wherein the first axial stiffness is different from the second axial stiffness, and wherein the first axial stiffness is preferably smaller than the second axial stiffness.

4. The guidewire according to claim 3, wherein
the first axial stiffness is in a range of 1.4 mN/mm to 30 mN/mm and/or wherein the second axial stiffness is in a range of 1.4 mN/mm to 30 mN/mm.

5. The guidewire according to claim 1, wherein
the first stiffness comprises a first lateral stiffness and the second stiffness comprises a second lateral stiffness, and wherein a range of the first lateral stiffness is different from a range of the second lateral stiffness.

6. The guidewire according to claim 5,
wherein the range of the first stiffness is 39 mN/mm to 2.28 N/mm, and/or
wherein the range of the second lateral stiffness is 1.75 N/mm to 2.28 N/mm.

7. The guidewire according to claim 1, wherein
the first portion comprises an atraumatic tip extending from the distal axial end, and wherein the atraumatic tip is preferably a J-tip.

8. The guidewire according to claim 7, wherein
the atraumatic tip has an axial stiffness in a range of 1.4 mN/mm to 30 mN/mm and/or a lateral stiffness of 39 mN/mm to 353 mN/mm.

9. The guidewire according to claim 7, wherein
the guidewire further comprises a sensor, wherein the sensor is disposed in or next to the first portion , and wherein the sensor is preferably disposed in a region between the atraumatic tip and the second portion.

10. The guidewire according to claim 9, wherein
further comprising a lead arrangement, wherein the lead arrangement is attached to the sensor, wherein the lead arrangement extends in axial direction to the proximal axial end of the elongated main body, and wherein the lead arrangement is preferably-coated or covered.

11. The guidewire according to claim 10, wherein
the elongated main body comprises a hollow portion, wherein the lead arrangement is preferably disposed within the hollow portion.

12. (canceled)

13. The guidewire according to claim 9, wherein
the sensor is disposed in a magnetically permeable housing, the housing comprising a plastic material.

14. The guidewire according to claim 9, wherein
the sensor is disposed within an extruded protection layer.

15. The guidewire according to claim 10, wherein
the guidewire comprises at least one further sensor, wherein the at least one further sensor is disposed at a certain distance from the distal axial end to the proximal axial end or is disposed a certain distance from the sensor disposed in the region between the atraumatic tip and the second portion or in the atraumatic tip, and wherein the certain distance is 10 cm.

16. (canceled)

17. The guidewire according to claim 15, wherein the lead arrangement is attached to the at least one further sensor.

18. The guidewire according to claim 15,
wherein the at least one further sensor is disposed in a magnetically permeable housing, the housing comprising a plastic material.

19. The guidewire according to claim 15, wherein
the at least one further sensor is disposed within an extruded protection layer.

20. The guidewire according to claim 15 9, wherein the sensor comprises a measurement coil, and wherein the measurement coil is directly wound onto a portion of the guidewire.

21. The guidewire according to claim 20, wherein the at least one further sensor comprises a measurement coil, and wherein the measurement coil is directly wound onto a portion of the guidewire.

22. The guidewire according to claim 1, wherein
the elongated main body further comprises at least one cardiac echo marker.
